(19) United States
(12) Patent Application Publication
(10) Pub. No.: US 2025/0066736 A1
(43) Pub. Date: Feb. 27, 2025

(54) HIGH-THROUGHPUT 3D CELL SPHEROID CULTURE CHIP, PREPARATION PROCESS AND USES THEREOF

(71) Applicant: National Taiwan University, TAIPEI CITY (TW)

(72) Inventors: Yung-Te Hou, Taipei City (TW); Tzong-jih Cheng, Taipei City (TW); Kai-Yi Cheng, Taipei City (TW); Richie L.C. Chen, Taipei City (TW); Po-Chuan Hsieh, Taipei City (TW); Hsien-Yi Hsiao, Taipei City (TW)

(21) Appl. No.: 18/812,540

(22) Filed: Aug. 22, 2024

Related U.S. Application Data
(60) Provisional application No. 63/534,588, filed on Aug. 25, 2023.

(51) Int. Cl. C12N 5/071; C12M 3/00; C12M 3/06

(52) U.S. Cl. CPC C12N 5/0671; C12M 21/08; C12M 23/16; C12N 2513/00; C12N 2533/90

(57) ABSTRACT

The present disclosure provides a high-throughput 3D cell spheroid culture chip, the preparation process and uses thereof. Through various efficacy experiments in the present disclosure, first evidence of using hydrogels derived from decellularized liver tissue as a self-healing biomaterial to reduce damage to damaged hepatocytes and enhance liver function in vitro is provided. Integrating endothelial cell-covered hepatocyte spheroids into DLM-CP hydrogels is a promising approach to develop microbial liver tissue, providing a potential solution for liver fibrosis recovery and promoting cell-level therapy. DLM-CP hydrogels show great potential for cell encapsulation for therapeutic purposes in future clinical settings and may be applied to ultra-high-throughput three-dimensional cell spheroid culture chips. It is used to create artificial tissues and organs, becoming a high-value tool widely used in biomedical research and pharmaceutical fields.

### CROSS-REFERENCE TO RELATED APPLICATION

This application claims priority of U.S. provisional application No. 63/534,588, filed on Aug. 25, 2023, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 113F0233-IE_Sequence_listing. The XML file is 10000 bytes; was created on Aug. 20, 2024.

### BACKGROUND OF THE INVENTION

### 1. Field of the Invention

The present invention relates to a high-throughput 3D cell spheroid culture chip, the preparation process and uses thereof.

### 2. The Prior Art

Three-dimensional cell spheroids can ensure better interactions between cells and cells and between cells and matrix, and can preserve the complete extracellular matrix when implanted in the body, which is helpful for the expression and play of cell functions. Current three-dimensional cell culture methods include cell suspension culture bottles, hanging drop methods and ultra-low adhesion culture dishes. However, these methods have shortcomings such as low spheroidization efficiency and inconvenient operation.

In order to solve the above-mentioned problems, those skilled in the art urgently need to develop a novel high-throughput 3D cell spheroid culture chip and the preparation process thereof for the benefit of a large group of people in need thereof.

### SUMMARY OF THE INVENTION

A primary objective of the present invention is to provide a high-throughput 3D cell spheroid culture chip, comprising a polycarbonate (PC) substrate, a plurality of probe arrays, and a plurality of cells, wherein the plurality of probe arrays are pressed into the polycarbonate substrate.

Another objective of the present invention is to provide a method for preparing the above mentioned high-throughput 3D cell spheroid culture chip, comprising the following steps: (a) pressing the plurality of probe arrays into the polycarbonate (PC) substrate to obtain a formed cell spheroid culture chip; (b) immersing the formed cell spheroid culture chip in a solution to obtain a solution-coated cell spheroid culture chip; and (c) inoculating the plurality of cells on the solution-coated cell spheroid culture chip, and culturing, followed by collecting spheroids, thereby preparing the high-throughput 3D cell spheroid culture chip.

According to an embodiment of the present invention, the plurality of probe arrays are pressed into the polycarbonate substrate with an imprint depth set to 500 μm.

According to an embodiment of the present invention, the high-throughput 3D cell spheroid culture chip is subjected to surface coating modification on the polycarbonate substrate with bovine serum albumin (BSA), 2-methacryloyloxyethyl phosphoryl choline (MPC), carboxymethylcellulose (CMC), or Pluronic-F127.

According to an embodiment of the present invention, each of the plurality of probe arrays is composed of 170 probes, and each probe has a diameter of 350 μm, so that each of the plurality of probe arrays forms a circular configuration with a diameter of 1 cm.

According to an embodiment of the present invention, the polycarbonate substrate is a circular polycarbonate substrate with a diameter of 3 cm, and three probe arrays are arranged on a diameter of the polycarbonate substrate.

According to an embodiment of the present invention, the high-throughput 3D cell spheroid culture chip further comprises a chitosan and phenol (CP)-based self-healing hydrogel, wherein the plurality of cells are a plurality of primary mature hepatocytes, and the plurality of primary mature hepatocytes are embedded in the CP-based self-healing hydrogel, thereby forming a hepatocyte spheroid.

According to an embodiment of the present invention, the high-throughput 3D cell spheroid culture chip further comprises an endothelial cell, wherein the endothelial cell covers the hepatocyte spheroid.

According to an embodiment of the present invention, the endothelial cell is a human umbilical vein endothelial cell (HUVEC).

According to an embodiment of the present invention, the CP-based self-healing hydrogel comprises a decellularized liver matrix (DLM).

According to an embodiment of the present invention, the CP-based self-healing hydrogel comprises difunctionalized polyethylene glycol (DP).

According to an embodiment of the present invention, the solution comprises a component selected from the group consisting of: bovine serum albumin (BSA), 2-methacryloyloxyethyl phosphoryl choline (MPC), carboxymethylcellulose (CMC), and Pluronic-F127.

Another objective of the present invention is to provide a high-throughput cell culture method, comprising using the above mentioned high-throughput 3D cell spheroid culture chip.

In summary, through the results illustrated in the following examples, the first evidence of using hydrogel derived from decellularized liver tissue and, serving as a self-healing biomaterial, to mitigate damage and augment hepatic functions in injured hepatocytes in vitro is presented. The integration of endothelial cells (ECs)-covered hepatocyte spheroids into the decellularized liver matrix-chitosan-phenol (DLM-CP) hydrogel is a promising approach for developing microbionic liver tissues, offering potential solutions for liver fibrosis recovery and promoting cellular-level therapies. The DLM-CP hydrogel shows considerable potential for future clinical use in cell encapsulation for therapeutic purposes. It may be applied to ultra-high-throughput three-dimensional (3D) cell spheroid culture chips to create artificial tissues and organs, becoming a high-value tool widely used in biomedical research and pharmaceutical fields.

In particular, the present invention uses polycarbonate (PC), which is cheaper, as a cell culture platform, and uses a self-made mold to engrave holes of the same well diameter and well depth on the PC. Cells can be evenly deposited in the holes to form 3D spheroids. Due to different types of cells, there would be corresponding optimal 3D spheroid sizes for subsequent growth. The present invention can use a very simple and rapid method to engrave a large number of holes with different well diameters and well depths, and can generate a large number of 3D cells of uniform size. Moreover, the present invention is compatible with standardized cell culture plate specifications, so it has advantages in efficiency, cost-effectiveness, data accuracy, scalability and versatility, and is believed to be more widely used in biomedical research. The PC material used in the present application is also suitable for various surface coating modifications to prevent cell attachment, such as common cell non-adhesive molecules including bovine serum albumin (BSA), 2-methacryloyloxyethyl phosphoryl choline (MPC), carboxymethylcellulose (CMC), Pluronic-F127, etc., providing further effective formation of 3D spheroids.

The 3D spheroid culture method used in the present invention does not need to add Matrigel® to the culture environment to promote cell differentiation, simply using physical methods (making holes and shaking culture) can achieve the effect of differentiating cells into 3D spheroids. Not only is it fast and simple, it can also significantly reduce the cost of use. Although cancer cell lines can be commonly used as in vitro validation platforms for drug testing and biomedical applications, there are still differences in function and gene expression between primary mature cells and cloned cancer cell lines. For example, in vitro culture platforms for liver cells generally use liver cancer cell lines such as HepG2 and HepRG, but primary mature hepatocytes are the gold standard that best matches the real situation in the body. Therefore, the exemplary cells used in the present invention are primary mature hepatocytes. Because primary mature hepatocytes still retain their normal cytokines and the ability to secrete extracellular matrix, they are different from the mechanism of 3D spheroids formed by liver cancer cell lines in vitro. For this reason, it is believed that the device made by the present invention is more suitable for mass production of uniform 3D spheroids of various cells.

The embodiments of the present invention would be further described below. The following examples are used to illustrate the present invention and are not intended to limit the scope of the present invention. Anyone skilled in the art can make some changes and modifications without departing from the spirit and scope of the present invention. Therefore, the scope of the present invention shall be defined by the appended claims.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 is a schematic of the fundamental concepts of the present invention.

FIGS. 2A-2B are schematics of the cell spheroid culture chip, in which

FIG. 2A in (a) Imprinting mold densely packed with probes, wherein the inner circle is the probe array; (b) Schematic of a hydraulic press machine; (c) Schematic of the prepared cell spheroid culture chip. When using a mold to imprint a culture chip, do not overlap the imprints, otherwise the grooves on the culture chip would be deformed. The directionality is not certain, and the main purpose is to make the imprinted parts evenly distributed in the circular polycarbonate substrate.

FIG. 3A shows procedures for decellularized porcine liver preparation.

FIG. 3B shows storage modulus (G′) and loss modulus (G″) of decellularized liver matrix-chitosan-phenol (DLM-CP) hydrogel versus gelling time at a frequency of 0.1 Hz and dynamic strain of 1%.

FIG. 3C shows strain-sweep analysis of DLM-CP hydrogel at dynamic strain amplitudes ranging from 1% to 500% and a frequency of 0.1 Hz, that is the shear strain-induced changes in the modulus of the DLM-CP hydrogel over the dynamic strain range of 1% to 500%.

FIG. 3D shows continuous rheological monitoring of DLM-CP hydrogel damage and healing under alternating dynamic strains of 1% and 400%, that is the reversible gel-sol-gel transition of the DLM-CP hydrogel under continuous strain-induced damage and subsequent healing cycles alternating at strains of 1% and 400%.

FIG. 4A in (a) shows formation and morphologies of cell spheroids. The microscopic examination of the cell spheroid culture chip revealed its transparent and translucent appearance before cell seeding ((a) of FIG. 4A). FIG. 4A in (b) shows hepatocytes inoculated on a cell spheroid culture chip. After cell seeding, the majority of cells settled in the culture chip probe array's cavities ((b) of FIG. 4A). FIG. 4A in (c) shows hepatocytes inoculated on the cell spheroid culture chip after 1 day. In detail, after 24 h of culture, hepatocytes began to attach at the periphery, ultimately forming the central core of the spheroids ((c) of FIG. 4A). FIG. 4A in (d) shows hepatocytes inoculated on the cell spheroid culture chip after 3 days. In detail, over time, the hepatocyte clusters grew in size through aggregation and adhesion with neighboring cells. By day 3 ((d) of FIG. 4A), these hepatocyte clusters had expanded to exceed a diameter of 100 μm.

FIG. 4B shows microscopic images of cell spheroids. ((e) of FIG. 4B) Petri dish (control), day 1. ((f) of FIG. 4B) Cell spheroid culture chip, day 1. On day 1, the cells in the Petri dish exhibited a dispersed distribution ((e) of FIG. 4B), whereas those on the cell spheroid culture chip aggregated within the chip's cavities ((f) of FIG. 4B). ((g) of FIG. 4B) Petri dish, day 3. ((h) of FIG. 4B) Cell spheroid culture chip, day 3. By day 3, the spheroids cultured on the cell spheroid culture chip clearly displayed higher uniformity in terms of size than those in the Petri dish ((g)-(h) of FIG. 4B). FlowCam images of spheroids on Petri dish and the cell spheroid culture chip. (i) Petri dish, day 3. (j) Cell spheroid culture chip, day 3.

FIG. 4C in (k) and (1) shows spheroid size distribution via FlowCam analysis. (k) Petri dish (control), day 3. (l) Cell spheroid culture chip, day 3. In FlowCam analysis ((i)-(j) of FIG. 4B), the cells in the Petri dish exhibited the following size distribution: 40.8% for 50-75 μm, 21.5% for 76-100 μm, 16.1% for 101-125 μm, and 11.6% for 126-150 μm ((k) of FIG. 4C). By contrast, the cells on the cell spheroid culture chip exhibited the following size distribution: 13.3% for 50-75 μm, 22.0% for 76-100 μm, 34.3% for 101-125 μm, and 17.1% for 126-150 μm ((1) of FIG. 4C).

FIG. 5A shows co-culture of hepatocyte spheroids with human umbilical vein endothelial cells (HUVECs). (a) Original hepatocyte spheroids. (b) Hepatocyte spheroids co-cultured with HUVECs after 1 day. (c) Hepatocyte spheroids co-cultured with HUVECs after 2 days. (d) Hepatocyte spheroids co-cultured with HUVECs after 3 days. Bar=100 μm.

FIG. 5B shows HUVEC-covered hepatocyte spheroids embedded in DLM-containing CP hydrogel after 3 days. (e) Picrosirius red staining analysis. (f) CD31 staining analysis. Bars=500 μm and 200 μm, separately. Moreover, due to the presence of DLM components in the CP hydrogel, the results of picrosirius red staining were positive for both the extracellular matrix (ECM) secreted by the cells and the surrounding hydrogel structure, indicating the effective diffusion of DLM within the CP hydrogel ((e) of FIG. 5B). Furthermore, the staining results for CD31 revealed a yellow staining pattern, indicative of CD31 expression ((f) of FIG. 5B).

FIG. 5C shows fluorescence images of (g-j) hepatocyte spheroids and (k-n) hepatocyte spheroids co-cultured with HUVECs after 3 days. (g, k) DAPI-staining cells. (h, 1) CK18-staining cells. (i, m) CD31-staining cells. (j, n) Merge images. Bars=200 μm.

FIG. 5D shows gene expression of hepatocyte spheroids and hepatocyte spheroids co-cultured with HUVECs after 1 day. (o) CYP1A2. (p) CYP3A23. (q) CYP3A4. ***p<0.001 vs. hepatocyte spheroids.

FIG. 6A shows Albumin synthesis of hepatocytes in DLM-CP hydrogel at various DLM concentrations (0, 0.02, 0.1, 0.5 g/L).

FIG. 6B shows Albumin gene expression of hepatocytes in DLM-CP hydrogel at various DLM concentrations (0, 0.02, 0.1, 0.5 g/L) after 14 days of culture. *p<0.05, ***p<0.001 vs. 0.02 g/L DLM group on days 3, 5, 7, 10, and 14, respectively.

FIG. 7A shows relative cell viability of hepatocytes with CCl₄-induced injury at different concentrations on days 1 and 3.

FIG. 7B shows that the injured hepatocytes cultured in the 0.02 g/L DLM-containing CP hydrogel (hepatocyte spheroids embedded in DLM-containing CP hydrogel (CDS), HUVEC-covered hepatocyte spheroids embedded in DLM-containing CP hydrogel (CDSH)) exhibited higher relative cell viability than those cultured in the CS hydrogel.

FIG. 7C shows urea synthesis of injured hepatocytes treated with CS, CDS, and CDSH after 7 days of culture.

FIG. 7D shows relative cytotoxicity of injured hepatocytes treated with CS, CDS, and CDHS after 7 days of culture. *p<0.05, **p<0.01, ***p<0.001 vs. CDSH group on days 1, 3, 5, and 7, respectively; #p<0.05, ##p<0.01, ###p<0.001 vs. CDS group on days 1, 3, 5, and 7, respectively.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

Unless otherwise stated in the context, “a”, “the” and similar terms used in the specification (especially in the following claims) should be understood as including singular and plural forms.

According to the present invention, the term “self healing” is a term well known to those with ordinary skills in the art, for example, see TWI697511.

According to the present invention, the term “high throughput” refers to the ability to form large numbers of cell spheroids.

The present invention is further illustrated by the following examples. These examples are provided for illustration only and are not intended to limit the scope of the present invention. The scope of the present invention is shown in the appended claims.

The chemicals and equipment are as follows. Phloretic acid [3-(4-hydroxyphenyl) propionic acid] were purchased from Alfa Aesar (Heysham, UK). Anhydrous THF (tetrahydrofuran), Diethyl ether were purchased from Echo (Miaoli, Taiwan). Chloroform (anhydrous, ≥99%, contains 0.5-1.0% ethanol as stabilizer) (288306-100ML), Carbon tetrachloride (anhydrous, ≥99.5%) (289116-1L) 1-(3-(dimethylcamino) propyl)-3-ethyl-carbodimide methiodide (EDC, L-165344-10G), N-hydroxysuccinimide (NHS; 130672-25G), 2-(N-morpholino) ethanesulfonic acid hydrate (MES hydrate, M3567), Lactate dehydrogenase activity assay kit (LDH; MAK066), Glutaraldehyde solution (50% in H₂O) (340885-25M), Triton X-100 (X-100), Carbon tetrachloride (anhydrous, ≥99.5%) (289116-1L), Dimethyl sulfoxide (DMSO; D4540), William's E medium (WE medium; SI-W4125), DMEM/F12 medium (CC113-0500), Hydrocortisone (H0888), Hydrochloric acid (ACS reagent, 37%) (320331-2.5L), Papain (P4762), Urethane (≥99%) (U2500-100G), L-Ascorbic acid (A8960-5G), L-Proline (P0380-100G), Dexamethasone (D4902-100 MG), 2-Mercaptoethanol (M3148), Bromophenol blue (B0126-25G), Pepsin from porcine gastric mucosa (≥250 units/mg solid), Acrylamide/Bis-acrylamide, 30% solution (A3699-100ML), TEMED (T9281-25ML), Glycine (410225-250G), Ammonium sulfate (A4915), Methanol (179337), Chitosan (50-190 kDa, 448869), 4-formylbenzoic acid, DCC (N,N′-dicyclohexylcarbodiimide), Poly(ethylene glycol) (PEG, average molecular weight 8 kDa, MES [2-(N-morpholino) ethanesulfonic acid hydrate, DMAP [4-(dimethylamino)pyridine] were purchased from Sigma-Aldrich (Munich, Germany). EGM™-2 (Endothelial Cell Growth Medium-2 BulletKit™, CC-3162) was purchased from LONZA (Basel, Switzerland). Cellmatrix Type I-A (Collagen, Type I, 3 mg/mL, pH 3.0), Trypan blue 0.4% (207-17081), Collagenase (034-22363), UltraPure DNase/RNase-Free Distilled Water (034-22363) were purchased from FUJIFILM Wako Pure Chemicals (Osaka, Japan). Penicillin-streptomycin (15140-122), 2-Propanol (29113-95), Nicotinamide (24317-72), Albumin, bovine serum (08587-42), NaCl (31320-05), KCl (28514-75), Sodium phosphate (317-18), Disodium phosphate (31723-35), Phenol red (26807-21), 2-Propanol (29113-95), Ethanol (95%) (147-11), Sodium dodecyl sulfate (SDS; 31607-65) were purchased from Nacalai (Kyoto, Japan). Epidermal growth factor (EGF, PMG8041), Fetal bovine serum (FBS, 26140-079) were purchased from GIBCO (Grand Island, NY, USA). Paraformaldehyde solution (4% in PBS) (J19943), Goat anti-Rabbit IgG (H+L) Highly cross-adsorbed secondary antibody, Alexa Fluor Plus 594 (A32740), Trypsin-EDTA, Insulin transferrin selenium solution (ITS-X, 100×, 41400045-10 mL), MTT assay (M6494) were purchased from Thermo Fisher Scientific (Waltham, MA, USA). Cell counting kit-8 (CCK-8, ALX-850-039-KI01) was purchased from Enzo (Farmingdale, New York, USA). Rat albumin ELISA quantitation kit (E110-125) were purchased from Bethyl Laboratories (Montgomery, TX, USA). DNA quantity Kit (PMC-AK06-COS) was purchased from COSMO Bio (Tokyo, Japan). QuantiChrom urea assay kit (BH08A23) were purchased from BioAssay Systems (Hayward, CA, USA). ReadyProbes cell viability imaging kit, Blue/Green (R37609) was purchased from Life Technologies (Carlsbad, CA, USA). Invitrogen TRIzol reagent (317112) was purchased from Invitrogen (Waltham, MA, USA). Acetic acid, Glacial (9508-01) were purchased from J. T. Baker (Phillipsburg, NJ, USA). PrimerScript 1^{st} strand cDNA Synthesis kit was purchased from TaKaRa (San Jose, CA, USA). Rotor-Gene SYBR Green PCR Kit (400) was purchased from QIAGEN (Germantown, MD, USA). qPCR primer (Albumin, GAPDH) were purchased from Genomics (Taipei, Taiwan). Attane (Isoflurane) was purchased from Panion & BF Biotech (Taipei, Taiwan). Entellan® (107961), Percoll® (GE17089101) were purchased from Merck (Branchburg, NJ, USA). Ethanol (100%) (32221) was purchased from Honeywell (North Carolina, USA). Hematoxyin and Eosin stain kit (HAE-1-IFU-RUO) was purchased from ScyTek (Utah, USA), Picrosirius red stain kit (TASS08-125) was purchased from BIOTnA (Kaohsiung, Taiwan). Rabbit polyclonal antibody recognizes CD31 (ARG52748) was purchased from Arigobio (Ontario, Canada). Tissue-Tek® O.C.T. compound (62550-01) was purchased from Sakura Finetek (Torrance, CA, USA). 10×PBS (CC704-0500) was purchased from Simply (Melbourne, Australia). Poly (2-methacryloyloxyethyl phosphorylcholine (MPC)-co-n-butyl methacrylate (BMA) (Lipidure®-CM5206) was purchased from NOF (Shibuya, Tokyo, Japan). The experimental protocol was reviewed and approved by the Ethics Committee on Animal Experiments of National Taiwan University (IACUC Approval No: NTU-110-EL-00123).

The procedure regarding preparation of chitosan-phenol (CP)-based self-healing hydrogels is as follows. The process of synthesizing CP and DP has been described in another study.^{[24]} In brief, 500 mg of chitosan (50-190 kDa) was dissolved in 20 mL of 0.25 N HCl. Subsequently, 25 mL of 100 mM MES was added, and the pH of the prepared chitosan solution was adjusted to 4.5 by using 1 N NaOH. Thereafter, 249.3 mg of phloretic acid was dissolved in 400 mL of 50 mM MES buffer, followed by the addition of 287.5 mg of EDC and 173 mg of NHS. The chitosan solution and phloretic acid solution were mixed and allowed to react for 24 h in the dark with stirring at room temperature. A dialysis membrane (molecular weight cutoff=12-14 kDa) was used to dialyze the product against distilled water. Solid chitosan-phenol (CP) was obtained through lyophilization.

We dissolved 2 g of PEG (8 kDa) in 100 mL of anhydrous THF. Subsequently, 375 mg of 4-formylbenzoic acid, 170 mg of DMAP, and 1 g of DCC (N,N′-dicyclohexylcarbodiimide) were sequentially added to the prepared PEG solution. The reaction was allowed to progress for 48 h at room temperature, after which the precipitate was removed. The resulting supernatant was precipitated in diethyl ether and subsequently dissolved in THE three times. After air drying, solid difunctionalized polyethylene glycol (DP) was obtained.

A 2.5 wt. % CP solution and 4 wt. % DP solution were obtained by separately dissolving CP and DP in ddH₂O. The final product (CP-based self-healing hydrogels) was prepared by mixing the prepared 2.5 wt. % CP and 4 wt. % DP solutions at a ratio of 1:1. The final concentration of both CP and DP in the CP-based self-healing hydrogels was 1%. Note that CP-based self-healing hydrogels were abbreviated CP hydrogels in the following text.

The procedure regarding rheological analysis is as follows. The viscoelasticity of the hydrogel was assessed using a rheometer (HR-2, TA Instruments, USA) with a 20-mm parallel plate geometry. The freshly prepared hydrogel was immediately added to the Peltier plate. The environmental temperature was maintained at 25° C. throughout the experiments. For time-dependent analysis, the storage moduli (G′) and loss moduli (G″) of the hydrogel were measured at a frequency of 0.1 Hz and a dynamic strain of 1%. The shear strain-induced modulus variations in DLM-CP hydrogels were investigated over a dynamic strain amplitude range of 1% to 500% and a frequency of 0.1 Hz. The self-healing property of the hydrogel was evaluated by subjecting the hydrogel to damage-healing cycles at alternate high (400%) and low (1%) dynamic strains at a frequency of 0.1 Hz for 120 s in each step.

The procedure regarding liver decellularization process is as follows. The experiment was conducted using commercially purchased pig liver, which was sectioned into small pieces measuring approximately 0.5 cm in length, width, and height. The liver fragments were subsequently immersed in PBS solution and agitated at 300 rpm to eliminate residual blood. Subsequently, the liver fragments were immersed in Triton X-100 and SDS solutions and agitated at 300 rpm to remove tissue fluid. Lastly, the fragments were washed with deionized water to eliminate any residual detergent. Following the decellularization and washing procedures, the resulting decellularized liver matrices (DLMs) were preserved in a 4° C. PBS solution containing 5% penicillin-streptomycin.

The procedure regarding isolation of hepatocytes and hepatocyte spheroid cultures is as follows. Mature hepatocytes were isolated from male Sprague Dawley rats through a two-step collagenase perfusion technique. The viability of hepatocytes was examined using the trypan blue exclusion method, with cell viability consistently exceeding 90% in all experiments. The cells were cultivated in a humidified incubator with 5% CO₂ at 37° C. Hepatocyte spheroids were cultured in William's E medium supplemented with 10% FBS, 1% penicillin-streptomycin, 1% insulin, transferrin, selenium, ITS-X, 50 μg/mL hydrocortisone, and 20 ng/mL EGF.

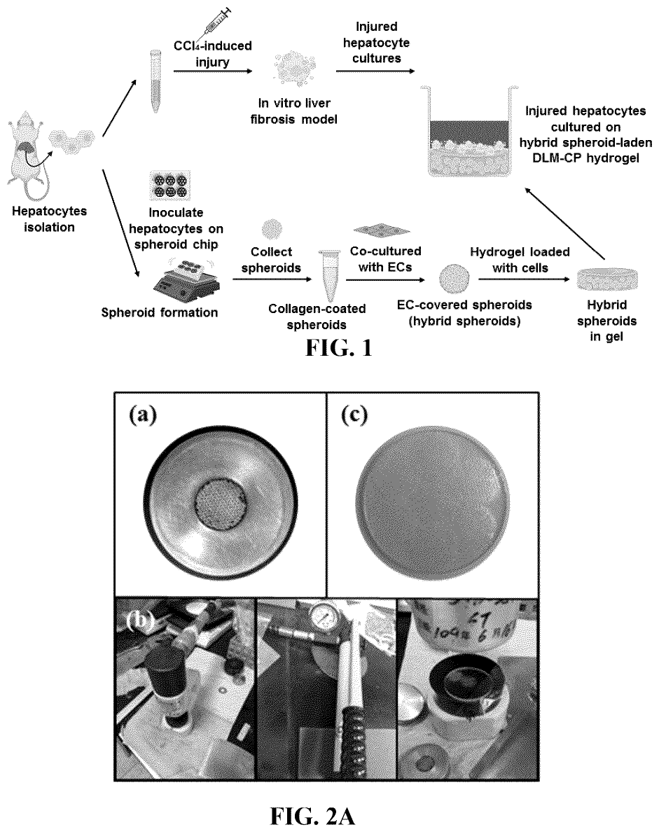

FIG. 1 is a schematic of the fundamental concepts of the present invention, see the description below for details.

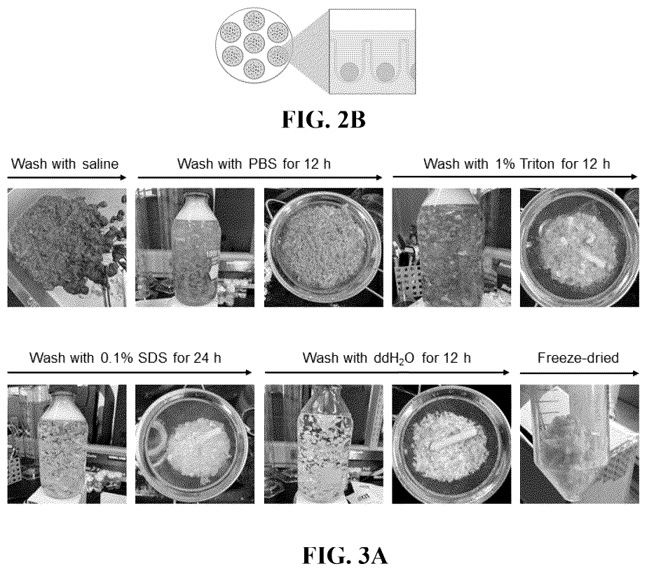

The cell spheroid culture chip was composed of a densely arranged array of probes (about 170 probes), each with a diameter of 350 μm, forming a probe array with a circular configuration with a diameter of 1 cm. The micromolding process involved pressing the probe array into a circular polycarbonate (PC) substrate measuring 3 cm in diameter by using a hydraulic press machine at a pressure of 20 MPa. The depth of the imprint was set at 500 μm, and the process was repeated seven times at different locations on the PC substrate to achieve a fully formed cell spheroid culture chip (see FIGS. 2A-2B). FIGS. 2A-2B are schematics of the cell spheroid culture chip, in which FIG. 2A in (a) Imprinting mold densely packed with probes, wherein the inner circle is the probe array; (b) Schematic of a hydraulic press machine; (c) Schematic of the prepared cell spheroid culture chip.

Subsequently, the cell spheroid culture chip was immersed in 500 μL of 2-methacryloyloxyethyl phosphoryl choline (MPC) solution (5 wt. % MPC dissolved in 75% ethanol) for 1 min, followed by washing with PBS. Hepatocytes (2.5 mL of 7.5×10⁵ cells/mL) were inoculated on the MPC-coated cell spheroid culture chip. The culture was placed on a 60-rpm orbital shaker to prevent cell adhesion. The culture medium was replaced on day 1, and spheroids were collected on day 3. That is to say, when the hepatocyte suspension is dropped on the culture chip, the liver cells would fall into the holes and grow, gradually forming cell spheres in the culture chip. The collected spheroids were filtered through a 40-μm cell strainer to eliminate single or dead cells. The diameter of the spheroids was determined using a FlowCam VS. (Fluid Imaging Technologies, Yarmouth, ME, USA) to obtain mass-produced cell spheroids.

The procedure regarding fabrication of human umbilical vein endothelial cell (HUVEC)-covered hepatocyte spheroids is as follows. The extracted hepatocyte spheroids were transferred to a 1.5-mL microcentrifuge tube and centrifuged at 50×g for 1 min at 25° C. Subsequently, the supernatant was aspirated, and hepatocyte spheroids were resuspended in 1 mL of collagen-coating solution, which was prepared by combining type I collagen, 10× culture medium (William's E medium supplemented with 1% ITS-X, 50 g/mL hydrocortisone, 20 ng/mL EGF, 10% FBS, and 1% PS), and reconstitution buffer at an 8:1:1 ratio, resulting in a final concentration of 1.2 mg/mL. The reconstitution buffer was prepared using 50 mM sodium hydroxide, 260 mM sodium hydrogen carbonate, and 200 mM HEPES. The resuspension was then incubated at 4° C. for 1 h. Following incubation, the spheroids were washed twice with the culture medium, and the spheroids were centrifugated at 50×g for 1 min at 4° C. The collagen coated spheroids were obtained by this procedure.

HUVECs, a representative endothelial cells (EC) commonly co-cultured with hepatocytes, were purchased from the Bioresource Collection and Research Center (H-UV001, Taiwan). HUVECs (3×10⁶ cells) of the 4 passage were co-cultured with 10,000 collagen-coated spheroids in a 0.1 mg/mL collagen-coated Petri dish measuring 90 mm in diameter. The co-culture process was conducted in a total volume of 12 mL, consisting of 6 mL of culture medium containing hepatocyte spheroids and 6 mL of endothelial growth medium-2 (EGM-2). The cells were then incubated at 37° C. with 5% CO₂ in an incubator, and the culture medium was replaced every 2 days.

The procedure regarding in vitro liver fibrosis model is as follows. To create an in vitro toxicity model, we used CCl₄ as a known hepatotoxic agent capable of inducing severe hepatotoxicity, ultimately leading to liver fibrosis. Initially, a stock solution of CCl₄ was prepared by dissolving it in DMSO to a concentration of 20%. Subsequently, hepatocytes were aliquoted into 15-mL centrifuge tubes. These hepatocytes were subjected to a 10-min toxication process with CCl₄ (0.05, 0.075, 0.1, 0.15, 0.2%)-containing culture medium (serum-free). Subsequently, hepatocytes were washed three times with PBS with centrifugation at 50×g for 1 min at 4° C. Thereafter, the supernatant was carefully removed, and the final cell pellet was seeded onto 0.1 mg/mL collagen-coated Petri dish at a density of 5×10⁵ cells/mL.

The procedure regarding HUVEC-covered hepatocyte spheroid-embedded gel cultures is as follows. Solid CP and DP were dissolved in PBS at concentrations of 2.5% w/v and 40% w/v, respectively. Then, 5 μL of DP solution was mixed with 10 μL of 10× William's E medium and combined with HUVEC-covered hepatocyte spheroids (1000 spheroids/gel). Different concentrations of DLM (0, 0.02, 0.1, 0.5 g/L) were then added to the mixture. PBS was added to the mixture to achieve a final volume of 50 μL, resulting in a final DP concentration of 4% w/v. Next, 50 μL of 2.5% w/v CP solution was thoroughly mixed with 50 μL of the aforementioned 4% w/v DP solution containing HUVEC-covered hepatocyte spheroids. This process yielded a 100-μL hydrogel precursor. The entire mixture was then injected into a 96-well plate. The CP hydrogel was formed within 1 min after mixing, after which 100 μL of culture medium was added. On the other hand, to exclude the influence of hepatocytes embedded within the gel, the concentration of hepatocytes spheroids within the CP hydrogel, hepatocytes spheroids within the DLM-containing CP hydrogel, and HUVEC-covered hepatocyte spheroids within the DLM-containing CP hydrogel were kept at 10,000 spheres/mL, separately. After a 30-min incubation period, the culture medium was replaced.

Next, 100 μL of CCl₄-injured hepatocytes (5× 10⁵ cells/mL) was inoculated into HUVEC-covered hepatocyte spheroids embedded in the DLM-containing CP hydrogel (CDSH). The culture medium was replaced every 24 h on the first day and then every 2 days thereafter.

The procedure regarding relative cell viability and liver-specific function of in vitro HUVEC-covered hepatocyte spheroids embedded in gel cultures is as follows. To assess relative cell viability, separate media formulations were used for 2D dish and 3D gel cultures. Specifically, 10% MTT-containing medium was used for the 2D dish culture, whereas 10% CCK8-containing medium was used for evaluating the cell viability in 3D hydrogel or spheroids. For MTT analysis, after 4-h incubation, half of the supernatant was removed from each well, and an equivalent volume of DMSO was added. Following an additional 10 min of incubation, absorbance was measured at 570 nm. For CCK8 analysis, after 4 h of incubation, 100 μL of the supernatant was transferred to a 96-well plate, and absorbance was measured at 450 nm. To calculate relative cell viability from days 1 to 14, the absorbance value of hepatocytes (or hepatocyte spheroids) in the control group (only gel without cells) measured on day 1 was used as baseline (blank) to exclude the influence of the gel. The absorbance value of each experimental condition was then divided by the value of the blank to obtain relative cell viability.

The culture medium was then collected for subsequent albumin and urea analyses. Albumin synthesis and urea synthesis are representative of hepatocyte-specific functions. The levels of albumin and urea in the media were determined using a rat albumin ELISA kit and urea assay kit, respectively. Absorbance (albumin: 450 nm; urea: 430 nm) was measured.

The procedure regarding hepatocyte toxicity is as follows. Lactate dehydrogenase (LDH) activity is a crucial marker of hepatocyte injury because abnormally elevated LDH activity is associated with liver diseases. To assess hepatocyte toxicity, LDH activity in the culture medium was quantified using a commercial kit. The culture media were collected as LDH samples after every 48-h medium change for the subsequent analysis of LDH activity. Absorbance was measured at 450 nm. To calculate the relative cytotoxicity from days 1 to 7, the absorbance value of normal hepatocytes in the WE medium containing 10% FBS on day 1 was used as blank. The absorbance value of each experimental condition was then divided by the value of this blank to obtain the relative cytotoxicity.

The procedure regarding histological analysis is as follows. Cryopreservation embedding was performed as follows: An initial layer of optimal cutting temperature (O.C.T) compound was spread within a tissue embedding box and cooled at −20° C. Subsequently, the hydrogel containing the cells was introduced at the center, after which the specimen was completely covered with O.C.T compound.

Hematoxylin and eosin (H&E) staining is a widely employed histological staining method, wherein hematoxylin stains the cell nuclei blue-purple and eosin stains the cytoplasm pink. H&E staining is used to observe cellular morphology. Furthermore, picrosirius red staining is a collagen-specific method in which the anionic dye picrosirius red binds to cationic collagen proteins, resulting in color variations that correspond to collagen abundance. This method enables the visualization of the collagen protein distribution in the specimen. Additionally, an increase in the number of CD31-positive cells is correlated with the HUVEC ratio in the cell constructs. These samples were then cooled and fixed and underwent slicing and staining at the Animal Experimental Center of the National Taiwan University Medical College.

The procedure regarding quantitative real-time PCR is as follows. Total RNA was extracted from the cells by using the TRIzol reagent following the manufacturer's protocol. The extracted RNA was reverse-transcribed into cDNA by using the PrimeScript first-strand cDNA synthesis kit. Quantitative real-time PCR was performed using a StepOne Real-Time PCR system. The following primers were used: F, 5′-TCCCAGACAAGGAGAAGCAG-3′ (SEQ ID NO:1) and R, 5′-TCACCGTCTTCAGCTGATCTT-3′ (SEQ ID NO:2) (96 bp, ALB); F, 5′-ATGGGCAAGCGCCGGTTGTAT-3′ (SEQ ID NO:3) and R, 5′-CAGTTGATGGAGAAGCGCAGCC-3′ (SEQ ID NO:4) (194 bp, CYP1A2); F, 5′-ATGTTCCCTGTCATCGAACAGTATG-3′ (SEQ ID NO:5) and R, 5′-TTCACAGGGACAGGTTTGCCT-3′ (SEQ ID NO:6) (80 bp, CYP3A23); F, 5′-AAGTCGCCTCGAAGATACACA-3′ (SEQ ID NO:7) and R, 5′-AAGGAGAGAACACTGCTCGTG-3′ (SEQ ID NO:8) (169 bp, CYP3A4); and F, 5′-GGCACAGTCAAGGCTGAGAATG-3′ (SEQ ID NO:9) and R, 5′-ATGGTGGTGAAGACGCCAGTA-3′ (SEQ ID NO:10) (143 bp, GAPDH). ALB, CYP1A2, CYP3A23, and CYP3A4 were used for determining hepatic functions and metabolic activity. The quantitative gene expression data were normalized to the expression levels of GAPDH, a housekeeping gene. Relative gene expression was analyzed using the ΔΔC_{q} method and was calculated as the fold change.

The procedure regarding immunohistochemical analysis is as follows. The cell suspension was collected, centrifuged at 4° C. (50 g, 1 min), and the supernatant was discarded. Then, 1 mL formalin was added for fixation. The following day, the sample underwent staining using the subsequent procedure: centrifugation at 4° C. (50 g, 1 min), removal of the supernatant, and washing three times with 1 mL PBS. After washing, 200 times diluted primary antibodies (CK-18, CD31) were added to the cell pellet. CK-18 stains hepatocytes, while CD31 stains endothelial cells. The mixture was incubated at 4° C. for 30 min, followed by two washes with 200 μL PBS. Subsequently, 400 times diluted secondary antibodies were added to the cell pellet, incubated in a dark environment at 4° C. for 30 min, and washed with 400 μL PBS twice. Finally, DAPI staining solution was added to the cell pellet, left for 15 min, and washed with 400 μL PBS twice. The stained cell pellet was placed in 500 μL PBS in a 24-well plate and left for imaging.

The procedure regarding statistical analysis is as follows. The results are presented as mean±standard deviation. Statistical analyses involving multiple samples were conducted using a two-tailed unpaired Student's t test or analysis of variance followed by a post hoc test, as appropriate. P<0.05 indicated statistical significance.

### Example 1

### Decellularized Liver Matrix (DLM) Preparation

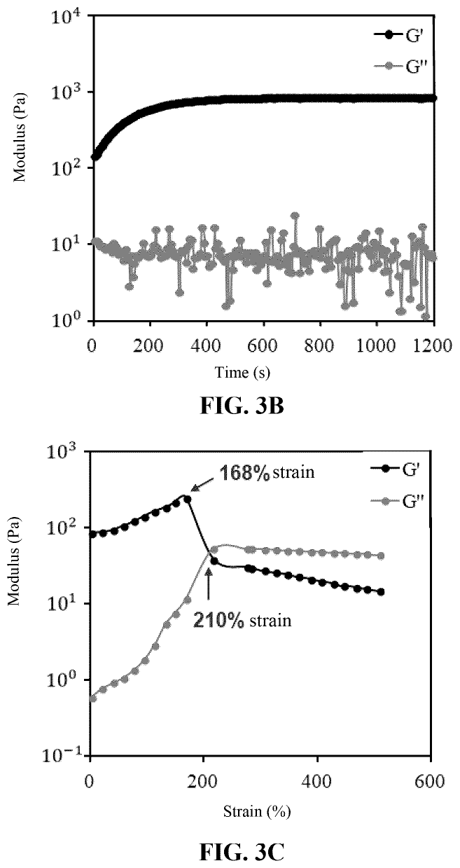

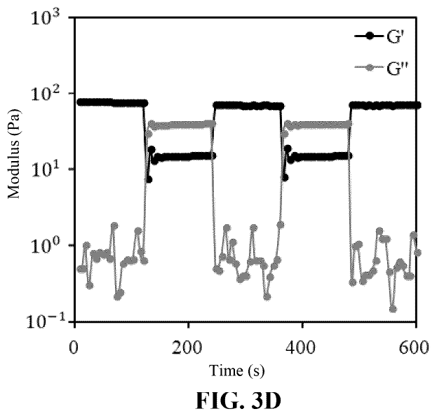

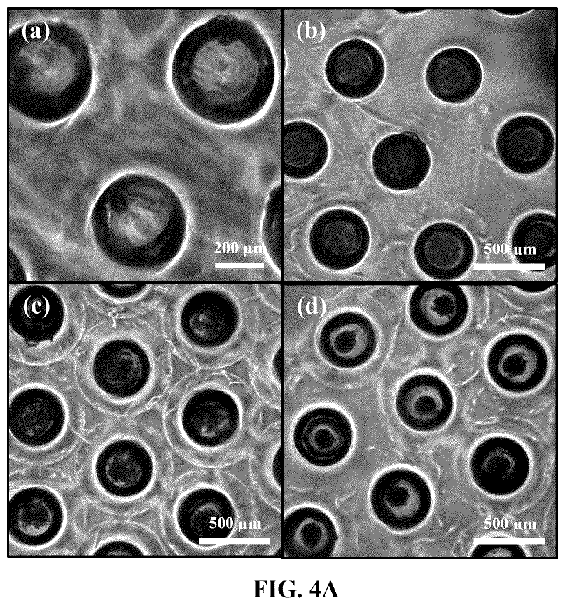

The decellularization process involved several steps. First, the porcine liver was finely minced into approximately 0.5 cm³ fragments. Subsequently, these fragments were sequentially immersed in PBS, Triton X-100, SDS, and ddH₂O to remove any remaining blood and eliminate any residual detergent. The resulting product was freeze-dried and minced to be the powder of decellularized liver matrix (DLM), as illustrated in FIG. 3A. This methodology is consistent with that described in previous literature (see Q. Wu, J. Bao, Y. J. Zhou, Y. J. Wang, Z. G. Du, Y. J. Shi, L. Li, H. Bu, Biomed. Res. Int. 2015, 2015, 785474).

### Example 2

### Rheology of DLM-CP Hydrogel

The storage shear modulus (G′) and loss shear modulus (G″) of DLM-CP hydrogel with respect to the gelling time are presented in FIG. 3B.

FIG. 3B shows storage modulus (G′) and loss modulus (G″) of decellularized liver matrix-chitosan-phenol (DLM-CP) hydrogel versus gelling time at a frequency of 0.1 Hz and dynamic strain of 1%. A remarkable increase in G′ was observed during the initial phase of gelation, after which G′ continued to increase slightly to a value of 845.43±6.42 Pa after 1200 s of gelling time.

FIG. 3C shows strain-sweep analysis of DLM-CP hydrogel at dynamic strain amplitudes ranging from 1% to 500% and a frequency of 0.1 Hz, that is the shear strain-induced changes in the modulus of the DLM-CP hydrogel over the dynamic strain range of 1% to 500%. The hydrogel exhibited a strain-hardening behavior between 1%-168% strain and a sudden reduction in the modulus when the strain exceeded 168%. Subsequently, the DLM-CP hydrogel displayed yielding behavior, transforming into a sol-like state (G′<G″) at higher strain values (>210%).

FIG. 3D shows continuous rheological monitoring of DLM-CP hydrogel damage and healing under alternating dynamic strains of 1% and 400%, that is the reversible gel-sol-gel transition of the DLM-CP hydrogel under continuous strain-induced damage and subsequent healing cycles alternating at strains of 1% and 400%.

During these alternating cycles, the DLM-CP hydrogel adopted a critical gel state (ready to flow) at a higher strain (400%) and rapidly switched to the initial gel state (G′>G″) at a lower strain (1%). These observations confirm the similarity of the DLM-CP hydrogel to the previously described self-healing CP hydrogel and its impressive capacity to fully recover the structural integrity after repeated cycles. This self-healing behavior makes the DLM-CP an injectable hydrogel and good implant candidate, which minimizes invasiveness—a highly desirable trait in implant applications. Furthermore, M. Ayyildiz, S. Cinoglu, C. Basdogan, J. Mech. Behav. Biomed. Mater. 2015, 49, 235-243 reported that the G′ and G″ values of liver samples ranging from 306 to 945 Pa and 56 to 150 Pa, respectively, at a frequency of 0.1 Hz. These findings align with the results depicted in FIG. 3B, indicating that the DLM-CP hydrogel possesses suitable hardness properties that render it compatible for use as a liver implant in therapeutic applications without causing damage.

### Example 3

### Hepatocyte Spheroid Cultures

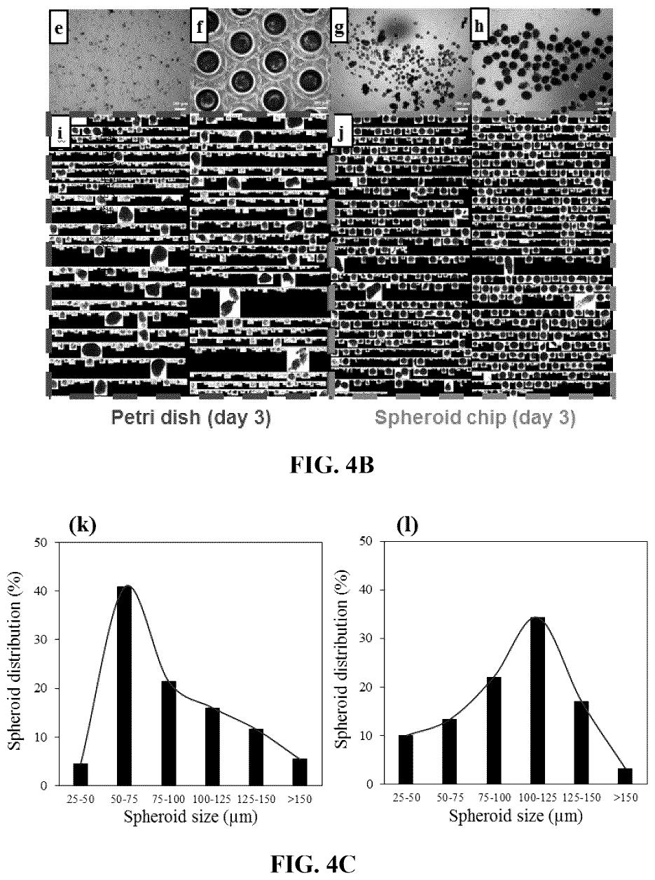

FIG. 4A in (a) shows formation and morphologies of cell spheroids. The microscopic examination of the cell spheroid culture chip revealed its transparent and translucent appearance before cell seeding ((a) of FIG. 4A). FIG. 4A in (b) shows hepatocytes inoculated on a cell spheroid culture chip. After cell seeding, the majority of cells settled in the culture chip probe array's cavities ((b) of FIG. 4A). FIG. 4A in (c) shows hepatocytes inoculated on the cell spheroid culture chip after 1 day. In detail, after 24 h of culture, hepatocytes began to attach at the periphery, ultimately forming the central core of the spheroids ((c) of FIG. 4A). FIG. 4A in (d) shows hepatocytes inoculated on the cell spheroid culture chip after 3 days. In detail, over time, the hepatocyte clusters grew in size through aggregation and adhesion with neighboring cells. By day 3 ((d) of FIG. 4A), these hepatocyte clusters had expanded to exceed a diameter of 100 μm. They displayed well-defined circular outlines with minimal observable boundaries between hepatocytes. We therefore harvested hepatocyte spheroids and conducted a particle size analysis to compare variations between spheroids cultured in 10-cm Petri dishes and those cultured on cell spheroid culture chips.

FIG. 4B shows microscopic images of cell spheroids. ((e) of FIG. 4B) Petri dish (control), day 1. ((f) of FIG. 4B) Cell spheroid culture chip, day 1. On day 1, the cells in the Petri dish exhibited a dispersed distribution ((e) of FIG. 4B), whereas those on the cell spheroid culture chip aggregated within the chip's cavities ((f) of FIG. 4B). ((g) of FIG. 4B) Petri dish, day 3. ((h) of FIG. 4B) Cell spheroid culture chip, day 3. By day 3, the spheroids cultured on the cell spheroid culture chip clearly displayed higher uniformity in terms of size than those in the Petri dish ((g)-(h) of FIG. 4B). FlowCam images of spheroids on Petri dish and the cell spheroid culture chip. (i) Petri dish, day 3. (j) Cell spheroid culture chip, day 3. FIG. 4C in (k) and (l) shows spheroid size distribution via FlowCam analysis. (k) Petri dish (control), day 3. (l) Cell spheroid culture chip, day 3. In FlowCam analysis ((i)-(j) of FIG. 4B), the cells in the Petri dish exhibited the following size distribution: 40.8% for 50-75 μm, 21.5% for 76-100 μm, 16.1% for 101-125 μm, and 11.6% for 126-150 μm ((k) of FIG. 4C). By contrast, the cells on the cell spheroid culture chip exhibited the following size distribution: 13.3% for 50-75 μm, 22.0% for 76-100 μm, 34.3% for 101-125 μm, and 17.1% for 126-150 μm ((1) of FIG. 4C). These results suggest that the use of the cell spheroid culture chip enhanced the size range of spheroids from 50-75 μm to the optimal size range of 101-125 μm, approaching the reported optimal range of 100 μm associated with optimal functionality (X. Zhu, Q. Wu, Y. He, M. Gao, Y. Li, W. Peng, S. Li, Y. Liu, R. Zhang, J. Bao, ACS Omega 2022, 7 (2), 2364-2376). The red curve represents the scatter plot with smooth line. Bars=200 μm and 500 μm, separately.

### Example 4

### HUVEC-Covered Hepatocyte Spheroid Cultures

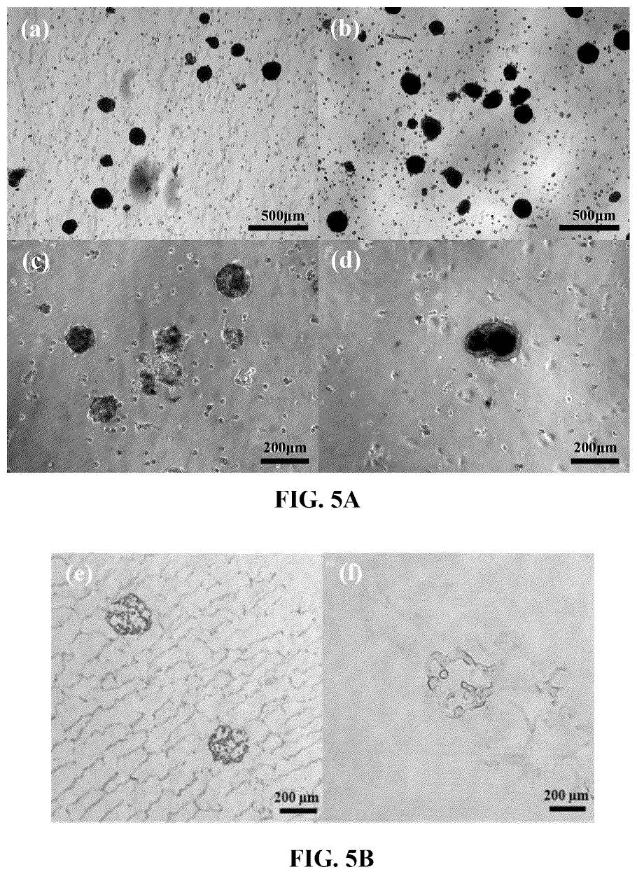

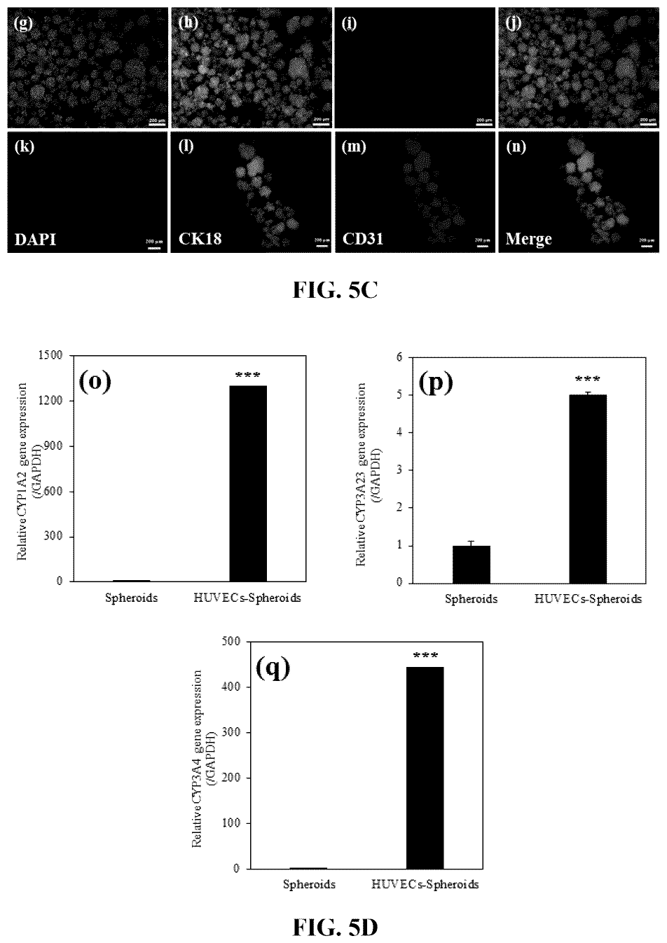

FIG. 5A shows co-culture of hepatocyte spheroids with human umbilical vein endothelial cells (HUVECs). (a) Original hepatocyte spheroids. (b) Hepatocyte spheroids co-cultured with HUVECs after 1 day. (c) Hepatocyte spheroids co-cultured with HUVECs after 2 days. (d) Hepatocyte spheroids co-cultured with HUVECs after 3 days. Bar=100 μm.

The original hepatocyte spheroids exhibited dark spherical morphology ((a) of FIG. 5A). However, after the introduction of HUVECs ((b) of FIG. 5A), a translucent outer ring appeared around the cell spheres ((c) of FIG. 5A), which eventually formed into a semitransparent membrane composed of HUVECs that coated the surface of hepatocyte spheroids ((d) of FIG. 5A).

FIG. 5B shows HUVEC-covered hepatocyte spheroids embedded in DLM-containing CP hydrogel after 3 days. (e) Picrosirius red staining analysis. (f) CD31 staining analysis. Bars=500 μm and 200 μm, separately. Moreover, due to the presence of DLM components in the CP hydrogel, the results of picrosirius red staining were positive for both the extracellular matrix (ECM) secreted by the cells and the surrounding hydrogel structure, indicating the effective diffusion of DLM within the CP hydrogel ((e) of FIG. 5B). Furthermore, the staining results for CD31 revealed a yellow staining pattern, indicative of CD31 expression ((f) of FIG. 5B). Notably, CD31 was predominantly present on the inner side of the sphere, suggesting the development of a vascular network promoted by HUVECs within hepatocyte spheroids on day 3. These results could be also explained by our fluorescence images.

FIG. 5C shows fluorescence images of (g-j) hepatocyte spheroids and (k-n) hepatocyte spheroids co-cultured with HUVECs after 3 days. (g, k) DAPI-staining cells. (h, l) CK18-staining cells. (i, m) CD31-staining cells. (j, n) Merge images. Bars=200 μm.

After 3 days of culture, CK18 expression, a marker of mature hepatocytes, was observed in both spheroids ((h) of FIG. 5C) and HUVEC-covered hepatocyte spheroids ((1) of FIG. 5C), indicating their hepatic functions. However, CD31 was not detected in the hepatocyte spheroids alone ((i) of FIG. 5C), whereas CD31 expression was evident around the HUVEC-covered hepatocyte spheroids ((m) of FIG. 5C). Consistent with the findings shown in (e)-(f) of FIG. 5B.

FIG. 5D shows gene expression of hepatocyte spheroids and hepatocyte spheroids co-cultured with HUVECs after 1 day. (o) CYP1A2. (p) CYP3A23. (q) CYP3A4. ***p<0.001 vs. hepatocyte spheroids.

On day 1, the relative gene expression levels of CYP1A2, CYP3A23, and CYP3A4 in the hepatocyte spheroids and HUVEC-covered hepatocyte spheroids were 1.00±0.74 and 1294.53±0.31 folds, 1.00±0.12 and 5.01±0.07 folds, 1.00±0.58 and 443.23±0.88 folds, respectively ((o)-(q) of FIG. 5D). The HUVEC-covered hepatocyte spheroids exhibited significantly greater CYP1A2, CYP3A23, and CYP3A4 gene expression than the hepatocyte spheroids, indicating the superior metabolic activity of HUVEC-covered hepatocyte spheroids.

### Example 5

### DLM-CP Hydrogel for Hepatocyte Cultures

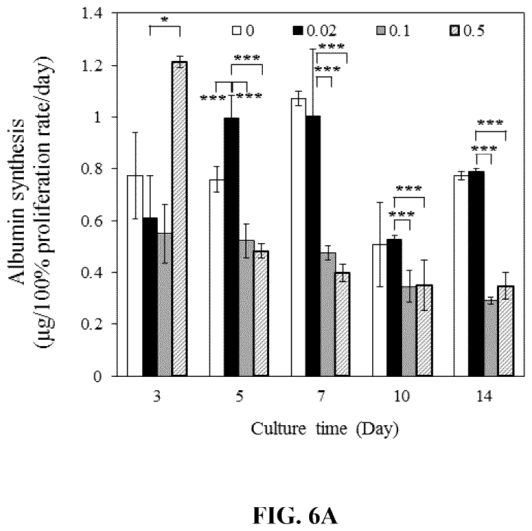

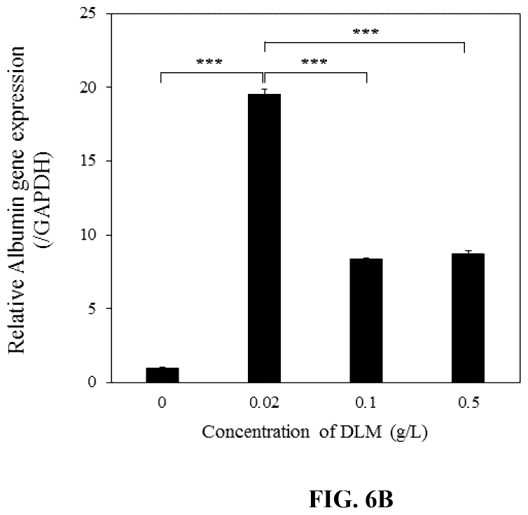

FIG. 6A shows Albumin synthesis of hepatocytes in DLM-CP hydrogel at various DLM concentrations (0, 0.02, 0.1, 0.5 g/L). As shown in FIG. 6A, on day 3, the group containing 0.5 g/L DLM exhibited a significantly higher albumin synthesis of 1.21±0.02 μg/100% proliferation rate/day compared with the DLM-free group (0.78±0.17 μg/100% proliferation rate/day). However, on days 5, 7, 10, and 14, this elevated synthesis gradually declined to 0.48±0.03, 0.40±0.04, 0.35±0.10, and 0.35±0.05 μg/100% proliferation rate/day, respectively, indicating a decrease in albumin synthesis over long-term culture. In the 0.1 g/L DLM-containing group, albumin synthesis on days 3, 5, 7, 10, and 14 was 0.55±0.11, 0.52±0.07, 0.47±0.03, 0.35±0.06, and 0.29±0.01 μg/100% proliferation rate/day, respectively, indicating suboptimal performance. In the 0.02 g/L DLM-containing group, albumin synthesis on day 3 was 0.61±0.16 μg/100% proliferation rate/day, which was slightly lower than that in the DLM-free group (0.78±0.17 μg/100% proliferation rate/day). However, on day 5, albumin synthesis in the DLM-containing group significantly increased to 1.00±0.09 μg/100% proliferation rate/day, denoting a significant difference compared with the DLM-free group (0.76±0.05 μg/100% proliferation rate/day). Although albumin synthesis did not continue to increase by day 7 (1.00±0.26 μg/100% proliferation rate/day), it remained close to that in the DLM-free group (1.07±0.03 μg/100% proliferation rate/day). On days 10 and 14, although no significant differences were noted between the DLM-free group and the 0.02 g/L DLM-containing group, a slight increase was still observed in the 0.02 g/L DLM-containing group.

FIG. 6B shows Albumin gene expression of hepatocytes in DLM-CP hydrogel at various DLM concentrations (0, 0.02, 0.1, 0.5 g/L) after 14 days of culture. *p<0.05, ***p<0.001 vs. 0.02 g/L DLM group on days 3, 5, 7, 10, and 14, respectively.

As shown in FIG. 6B, on day 14, the relative ALB gene expression levels in the DLM-free group and the DLM-containing groups (0.02, 0.1, 0.5 g/L) were 1.00±0.03, 19.54±0.30, 8.39±0.08, and 8.74±0.20 folds, respectively. The DLM-containing groups exhibited significantly higher ALB gene expression than the DLM-free group. These results suggest that hepatocyte spheroids encapsulated in CP hydrogels containing 0.02 g/L DLM exhibited the highest hepatic function, consistent with the findings presented in FIG. 6A. Therefore, the 0.02 g/L DLM-containing CP hydrogel was used in subsequent experiments.

### Example 6

### Hepatocyte Injury Conditions of CCl₄ and Recovery Effect of HUVEC-Covered Hepatocyte Spheroids Embedded in DLM-Containing CP Hydrogel (CDSH)

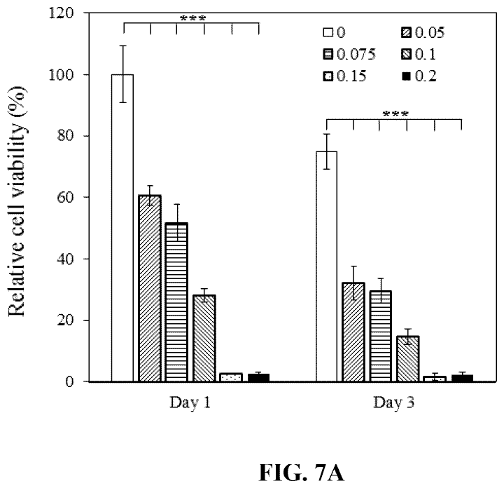

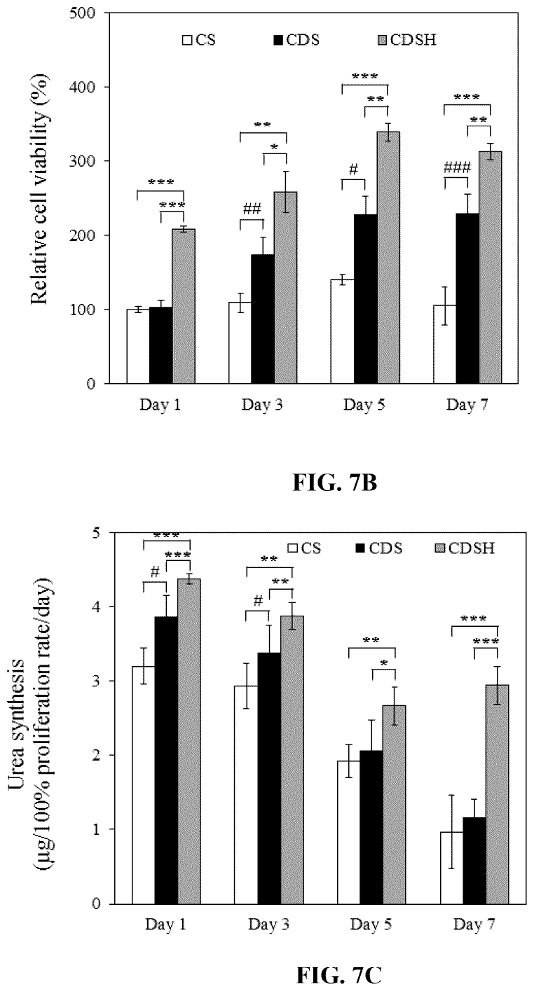

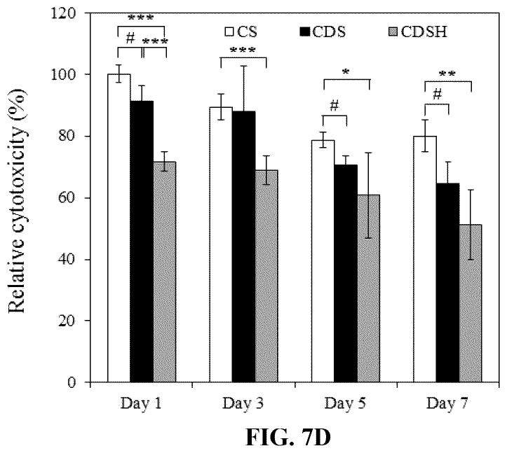

FIG. 7A shows relative cell viability of hepatocytes with CCl₄-induced injury at different concentrations on days 1 and 3. ***p<0.001 vs. no CCl₄-induced injury group on days 1, and 3, respectively. When exposed to a CCl₄ concentration of 0.075% for 24 h, cell viability reduced to approximately 50% of the original level (51.70%±5.99%). Toxicity was slightly lower at a CCl₄ concentration of 0.05% (60.53%±3.19%). However, higher CCl₄ concentrations yielded significantly higher toxicity (0.1%: 28.06%±2.29%, 0.15%: 2.67%±0.25%, 0.2%: 2.46%±0.63%, respectively). A similar trend was observed after 3 days of culture. These findings indicate the concentration of 0.075% CCl₄ to be the half-maximal inhibitory concentration (IC₅₀) for hepatocytes. Therefore, this concentration was selected in subsequent hepatocyte toxicity experiments.

The results in FIG. 7B indicate that the injured hepatocytes cultured in the 0.02 g/L DLM-containing CP hydrogel (CDS, CDSH) exhibited higher relative cell viability than those cultured in the CS hydrogel. On day 1, the relative cell viability in the CDSH group (208.05%±4.80%) was significantly higher than that in the CS group (100%±4.24%). On day 3, the relative cell viability of the CS group (108.78%±13.02%) was slightly higher than that noted on day 1, whereas the relative cell viability of the CDS and CDSH groups was 172.93%±23.57% and 258.29%±27.85%, respectively. On day 5, the relative cell viability of the CDS and CDSH groups was 227.80%±25.37% and 338.29%±12.77%, respectively, which was 2-3.5 times higher than that observed in the CS group. On day 7, the relative cell viability of the CDS and CDSH groups was 229.02%±25.99% and 311.95%±11.34%, respectively, which was still 2-3 times higher than that observed in the CS group. Overall, the CDS and CDSH groups exerted a beneficial effect on the relative cell viability of injured hepatocytes.

As shown in FIG. 7C, on day 1, urea synthesis in injured hepatocytes in the CS, CDS, and CDSH groups was 3.20±0.24, 3.86±0.29, and 4.38±0.07 μg/100% proliferation rate/day, respectively. On day 3, urea synthesis in the CS, CDS, and CDSH groups was 2.93±0.3, 3.37±0.38, and 3.88±0.18 μg/100% proliferation rate/day, respectively. On day 5, urea synthesis in the CS, CDS, and CDSH groups was 1.92±0.22, 2.06±0.42, and 2.67±0.25 μg/100% proliferation rate/day, respectively, and on day 7, urea synthesis was 0.97±0.50, 1.16±0.24, and 2.94±0.26 μg/100% proliferation rate/day, respectively. Despite the decreasing trend in urea synthesis over time, the CDSH group demonstrated a promising outcome on days 1-7, achieving 91% of the normal urea synthesis level (observed in the CS group on day 1) after 7 days of culture. This observation suggests a potential trend toward the restoration of impaired liver cell function when the cells were cultured in the DLM-containing hydrogel.

FIG. 7D shows relative cytotoxicity of injured hepatocytes treated with CS, CDS, and CDHS after 7 days of culture. As shown in FIG. 7D, on day 1, the CDS and CDSH groups exhibited relative cytotoxicity values of 91.23%±5.16% and 71.66%±3.11%, respectively, both of which were lower than that observed in the CS group (100%±2.81%). On day 3, the CS group exhibited a relative cytotoxicity of 89.39%±4.05%, which was slightly lower than the value obtained on day 1. The CDS and CDSH groups displayed relative cytotoxicity values of 88.05%±14.54% and 68.87%±4.54%, respectively. Notably, the relative cytotoxicity of the CDSH group remained significantly lower than that of the CS group. On day 5, the CS group exhibited a 22% decrease in relative cytotoxicity, attaining a value of 78.65%±2.51%. The CDS and CDSH groups demonstrated lower relative cytotoxicity values of 70.39%±3.07% and 60.74%±13.88%, respectively. On day 7, the CS group displayed relative cytotoxicity values of 79.9%±5.19%, whereas the CDS and CDSH groups exhibited lower relative cytotoxicity values of 64.55%±6.97% and 51.21%±11.49%, respectively, indicating a continued decrease in cytotoxicity. These results indicate that the CS group did not exhibit significant improvements in relative cell viability and urea synthesis within the toxic liver cell model. However, the combination of the DLM-CP hydrogel and hepatocyte spheroids (CDS) effectively enhanced the proliferation rate, hepatic function, and cytotoxicity inhibition in injured hepatocytes. Furthermore, these positive effects were further amplified when DLM-CP was combined with HUVEC-covered hepatocyte spheroids (CDSH).

In summary, through the results illustrated in the above examples, the first evidence of using hydrogel derived from decellularized liver tissue and, serving as a self-healing biomaterial, to mitigate damage and augment hepatic functions in injured hepatocytes in vitro is presented. The integration of endothelial cells (ECs)-covered hepatocyte spheroids into the DLM-CP hydrogel is a promising approach for developing microbionic liver tissues, offering potential solutions for liver fibrosis recovery and promoting cellular-level therapies. The DLM-CP hydrogel shows considerable potential for future clinical use in cell encapsulation for therapeutic purposes. It may be applied to ultra-high-throughput three-dimensional (3D) cell spheroid culture chips to create artificial tissues and organs, becoming a high-value tool widely used in biomedical research and pharmaceutical fields.

In particular, the present invention uses polycarbonate (PC), which is cheaper, as a cell culture platform, and uses a self-made mold to engrave holes of the same well diameter and well depth on the PC. Cells can be evenly deposited in the holes to form 3D spheroids. Due to different types of cells, there would be corresponding optimal 3D spheroid sizes for subsequent growth. The present invention can use a very simple and rapid method to engrave a large number of holes with different well diameters and well depths, and is believed to be more widely used in biomedical research. The PC material used in the present application is also suitable for various surface coating modifications to prevent cell attachment, such as common cell non-adhesive molecules including bovine serum albumin (BSA), 2-methacryloyloxyethyl phosphoryl choline (MPC), carboxymethylcellulose (CMC), Pluronic-F127, etc., providing further effective formation of 3D spheroids.

The 3D spheroid culture method used in the present invention does not need to add Matrigel® to the culture environment to promote cell differentiation, simply using physical methods (making holes and shaking culture) can achieve the effect of differentiating cells into 3D spheroids. Not only is it fast and simple, it can also significantly reduce the cost of use. Although cancer cell lines can be commonly used as in vitro validation platforms for drug testing and biomedical applications, there are still differences in function and gene expression between primary mature cells and cloned cancer cell lines. For example, in vitro culture platforms for liver cells generally use liver cancer cell lines such as HepG2 and HepRG, but primary mature hepatocytes are the gold standard that best matches the real situation in the body. Therefore, the exemplary cells used in the present invention are primary mature hepatocytes. Because primary mature hepatocytes still retain their normal cytokines and the ability to secrete extracellular matrix, they are different from the mechanism of 3D spheroids formed by liver cancer cell lines in vitro. For this reason, it is believed that the device made by the present invention can be more suitable for various types of cells to form uniform 3D spheroids.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A high-throughput 3D cell spheroid culture chip, comprising a polycarbonate (PC) substrate, a plurality of probe arrays, and a plurality of cells, wherein the plurality of probe arrays are pressed into the polycarbonate substrate.

2. The high-throughput 3D cell spheroid culture chip according to claim 1, wherein the plurality of probe arrays are pressed into the polycarbonate substrate with an imprint depth set to 500 μm.

3. The high-throughput 3D cell spheroid culture chip according to claim 1, which is subjected to surface coating modification on the polycarbonate substrate with bovine serum albumin (BSA), 2-methacryloyloxyethyl phosphoryl choline (MPC), carboxymethylcellulose (CMC), or Pluronic-F127.

4. The high-throughput 3D cell spheroid culture chip according to claim 2, wherein each of the plurality of probe arrays is composed of 170 probes, and each probe has a diameter of 350 μm, so that each of the plurality of probe arrays forms a circular configuration with a diameter of 1 cm.

5. The high-throughput 3D cell spheroid culture chip according to claim 2, wherein the polycarbonate substrate is a circular polycarbonate substrate with a diameter of 3 cm, and three probe arrays are arranged on a diameter of the polycarbonate substrate.

6. The high-throughput 3D cell spheroid culture chip according to claim 1, further comprising a chitosan and phenol (CP)-based self-healing hydrogel, wherein the plurality of cells are a plurality of primary mature hepatocytes, and the plurality of primary mature hepatocytes are embedded in the CP-based self-healing hydrogel, thereby forming a hepatocyte spheroid.

7. The high-throughput 3D cell spheroid culture chip according to claim 6, further comprising an endothelial cell, wherein the endothelial cell covers the hepatocyte spheroid.

8. The high-throughput 3D cell spheroid culture chip according to claim 7, wherein the endothelial cell is a human umbilical vein endothelial cell (HUVEC).

9. The high-throughput 3D cell spheroid culture chip according to claim 6, wherein the CP-based self-healing hydrogel comprises a decellularized liver matrix (DLM).

10. The high-throughput 3D cell spheroid culture chip according to claim 6, wherein the CP-based self-healing hydrogel comprises difunctionalized polyethylene glycol (DP).

11. A method for preparing the high-throughput 3D cell spheroid culture chip according to claim 1, comprising the following steps:
(a) pressing the plurality of probe arrays into the polycarbonate (PC) substrate to obtain a formed cell spheroid culture chip;
(b) immersing the formed cell spheroid culture chip in a solution to obtain a solution-coated cell spheroid culture chip; and
(c) inoculating the plurality of cells on the solution-coated cell spheroid culture chip, and culturing, followed by collecting spheroids, thereby preparing the high-throughput 3D cell spheroid culture chip.

12. The method according to claim 11, wherein the solution comprises a component selected from the group consisting of: bovine serum albumin (BSA), 2-methacryloyloxyethyl phosphoryl choline (MPC), carboxymethylcellulose (CMC), and Pluronic-F127.

13. The method according to claim 11, wherein the high-throughput 3D cell spheroid culture chip comprises a chitosan and phenol (CP)-based self-healing hydrogel, the plurality of cells are a plurality of primary mature hepatocytes, and the plurality of primary mature hepatocytes are embedded in the CP-based self-healing hydrogel, thereby forming a hepatocyte spheroid.

14. The method according to claim 13, wherein the high-throughput 3D cell spheroid culture chip further comprises an endothelial cell, and the endothelial cell covers the hepatocyte spheroid.

15. The method according to claim 13, wherein the CP-based self-healing hydrogel comprises a decellularized liver matrix (DLM).

16. A high-throughput cell culture method, comprising using the high-throughput 3D cell spheroid culture chip according to claim 1.


SEQUENCE LISTING (10 sequences)

SEQ ID NO: 1 - DNA, 20 bp, synthetic construct
  source 1..20: mol_type = other DNA
tcccagacaaggagaagcag

SEQ ID NO: 2 - DNA, 21 bp, synthetic construct
  source 1..21: mol_type = other DNA
tcaccgtcttcagctgatctt

SEQ ID NO: 3 - DNA, 21 bp, synthetic construct
  source 1..21: mol_type = other DNA
atgggcaagcgccggttgtat

SEQ ID NO: 4 - DNA, 22 bp, synthetic construct
  source 1..22: mol_type = other DNA
cagttgatggagaagcgcagcc

SEQ ID NO: 5 - DNA, 25 bp, synthetic construct
  source 1..25: mol_type = other DNA
atgttccctgtcatcgaacagtatg

SEQ ID NO: 6 - DNA, 21 bp, synthetic construct
  source 1..21: mol_type = other DNA
ttcacagggacaggtttgcct

SEQ ID NO: 7 - DNA, 21 bp, synthetic construct
  source 1..21: mol_type = other DNA
aagtcgcctcgaagatacaca

SEQ ID NO: 8 - DNA, 21 bp, synthetic construct
  source 1..21: mol_type = other DNA
aaggagagaacactgctcgtg

SEQ ID NO: 9 - DNA, 22 bp, synthetic construct
  source 1..22: mol_type = other DNA
ggcacagtcaaggctgagaatg

SEQ ID NO: 10 - DNA, 21 bp, synthetic construct
  source 1..21: mol_type = other DNA
atggtggtgaagacgccagta